SEQUENCE LISTING (63 sequences)

SEQ ID NO: 1 - protein, 15 aa, Artificial
  Synthetic Peptide
CHKFPCRDPQQYLFC

SEQ ID NO: 2 - protein, 15 aa, Artificial
  Synthetic Peptide
CTSPMCYVLKHQNRC

SEQ ID NO: 3 - protein, 15 aa, Artificial
  Synthetic Peptide
CTRPWCHSLLPRATC

SEQ ID NO: 4 - protein, 15 aa, Artificial
  Synthetic Peptide
CGRQFCHTLMPRHLC

SEQ ID NO: 5 - protein, 15 aa, Artificial
  Synthetic Peptide
CVRSHCSSLLPRIHC

SEQ ID NO: 6 - protein, 13 aa, Artificial
  Synthetic Peptide
CGREELPCRIKLC

SEQ ID NO: 7 - protein, 13 aa, Artificial
  Synthetic Peptide
CLRSYNLCPRINC

SEQ ID NO: 8 - protein, 15 aa, Artificial
  Synthetic Peptide
CHRDFPRCTWETQWC

SEQ ID NO: 9 - protein, 15 aa, Artificial
  Synthetic Peptide
CAPILCRWAERQGYC

SEQ ID NO: 10 - protein, 15 aa, Artificial
  Synthetic Peptide
CNAVLCSWARANSFC

SEQ ID NO: 11 - protein, 15 aa, Artificial
  Synthetic Peptide
  Xaa (8)..(8): Xaa is 1Nal
CNAVLCSXARANSFC

SEQ ID NO: 12 - protein, 15 aa, Artificial
  Synthetic Peptide
  Xaa (8)..(8): Xaa is 2Nal
CNAVLCSXARANSFC

SEQ ID NO: 13 - protein, 15 aa, Artificial
  Synthetic Peptide
  Xaa (9)..(9): Xaa is Aib
CNAVLCSWXRANSFC

SEQ ID NO: 14 - protein, 15 aa, Artificial
  Synthetic Peptide
  Xaa (10)..(10): Xaa is HArg
CNAVLCSWAXANSFC

SEQ ID NO: 15 - protein, 12 aa, Artificial
  Synthetic Peptide
CLELYQCWRGKC

SEQ ID NO: 16 - protein, 12 aa, Artificial
  Synthetic Peptide
CPSQYKCWRGKC

SEQ ID NO: 17 - protein, 12 aa, Artificial
  Synthetic Peptide
CLEVYKCWRGKC

SEQ ID NO: 18 - protein, 16 aa, Artificial
  Synthetic Peptide
  Xaa (4)..(4): Xaa is Aib
CANXVLCSWARANSFC

SEQ ID NO: 19 - protein, 15 aa, Artificial
  Synthetic Peptide
  Xaa (10)..(10): Xaa is Arg(Me)
CNAVLCSWAXANSFC

SEQ ID NO: 20 - protein, 15 aa, Artificial
  Synthetic Peptide
CNKVLCSWASRNSMC

SEQ ID NO: 21 - protein, 15 aa, Artificial
  Synthetic Peptide
CNQVLCRWAQVNSMC

SEQ ID NO: 22 - protein, 15 aa, Artificial
  Synthetic Peptide
CNRVLCAWATANSMC

SEQ ID NO: 23 - protein, 15 aa, Artificial
  Synthetic Peptide
CNTVLCNWARYNSLC

SEQ ID NO: 24 - protein, 15 aa, Artificial
  Synthetic Peptide
CNTVLCAWARANSYC

SEQ ID NO: 25 - protein, 15 aa, Artificial
  Synthetic Peptide
CNTVLCGWARANSLC

SEQ ID NO: 26 - protein, 15 aa, Artificial
  Synthetic Peptide
CNTVLCAWAMNNSMC

SEQ ID NO: 27 - protein, 15 aa, Artificial
  Synthetic Peptide
CNTVLCAWATQNSLC

SEQ ID NO: 28 - protein, 15 aa, Artificial
  Synthetic Peptide
CSPVLCAWATRNSLC

SEQ ID NO: 29 - protein, 15 aa, Artificial
  Synthetic Peptide
  Xaa (4)..(4): Xaa is tBuGly
CNAXLCSWARANSFC

SEQ ID NO: 30 - protein, 15 aa, Artificial
  Synthetic Peptide
  Xaa (4)..(4): Xaa is tBuAla
CNAXLCSWARANSFC

SEQ ID NO: 31 - protein, 15 aa, Artificial
  Synthetic Peptide
  Xaa (5)..(5): Xaa is tBuGly
CNAVXCSWARANSFC

SEQ ID NO: 32 - protein, 15 aa, Artificial
  Synthetic Peptide
  Xaa (5)..(5): Xaa is tBuAla
CNAVXCSWARANSFC

SEQ ID NO: 33 - protein, 15 aa, Artificial
  Synthetic Peptide
  Xaa (5)..(5): Xaa is Cba
CNAVXCSWARANSFC

SEQ ID NO: 34 - protein, 15 aa, Artificial
  Synthetic Peptide
  Xaa (14)..(14): Xaa is 2MePhe
CNAVLCSWARANSXC

SEQ ID NO: 35 - protein, 15 aa, Artificial
  Synthetic Peptide
  Xaa (14)..(14): Xaa is 3MePhe
CNAVLCSWARANSXC

SEQ ID NO: 36 - protein, 15 aa, Artificial
  Synthetic Peptide
  Xaa (14)..(14): Xaa is 4MePhe
CNAVLCSWARANSXC

SEQ ID NO: 37 - protein, 15 aa, Artificial
  Synthetic Peptide
  Xaa (14)..(14): Xaa is 2ClPhe
CNAVLCSWARANSXC

SEQ ID NO: 38 - protein, 15 aa, Artificial
  Synthetic Peptide
  Xaa (14)..(14): Xaa is 3ClPhe
CNAVLCSWARANSXC

SEQ ID NO: 39 - protein, 15 aa, Artificial
  Synthetic Peptide
  Xaa (14)..(14): Xaa is 4ClPhe
CNAVLCSWARANSXC

SEQ ID NO: 40 - protein, 15 aa, Artificial
  Synthetic Peptide
  Xaa (14)..(14): Xaa is 2FPhe
CNAVLCSWARANSXC

SEQ ID NO: 41 - protein, 15 aa, Artificial
  Synthetic Peptide
  Xaa (14)..(14): Xaa is 3FPhe
CNAVLCSWARANSXC

SEQ ID NO: 42 - protein, 15 aa, Artificial
  Synthetic Peptide
  Xaa (10)..(10): Xaa is Agb
CNAVLCSWAXANSFC

SEQ ID NO: 43 - protein, 15 aa, Artificial
  Synthetic Peptide
CNAVLCSWARANSYC

SEQ ID NO: 44 - protein, 15 aa, Artificial
  Synthetic Peptide
  Xaa (3)..(3): Xaa is HyP
  Xaa (10)..(10): Xaa is HArg
CNXVLCSWAXANSFC

SEQ ID NO: 45 - protein, 15 aa, Artificial
  Synthetic Peptide
  Xaa (10)..(10): Xaa is HArg
CNAVLCSWAXANSFC

SEQ ID NO: 46 - protein, 15 aa, Artificial
  Synthetic Peptide
  Xaa (10)..(10): Xaa is HArg
CNDVLCSWAXANSFC

SEQ ID NO: 47 - protein, 15 aa, Artificial
  Synthetic Peptide
  Xaa (10)..(10): Xaa is HArg
CNAVLCSWAXANSFC

SEQ ID NO: 48 - protein, 15 aa, Artificial
  Synthetic Peptide
  Xaa (14)..(14): Xaa is Hse(Me)
CNRVLCAWATANSXC

SEQ ID NO: 49 - protein, 15 aa, Artificial
  Synthetic Peptide
  Xaa (3)..(3): Xaa is HArg
  Xaa (10)..(10): Xaa is HArg
CNXVLCSWAXANSFC

SEQ ID NO: 50 - protein, 15 aa, Artificial
  Synthetic Peptide
  Xaa (10)..(10): Xaa is HArg
CNTVLCGWAXANSLC

SEQ ID NO: 51 - protein, 15 aa, Artificial
  Synthetic Peptide
CNTVLCGWARANSLC

SEQ ID NO: 52 - protein, 15 aa, Artificial
  Synthetic Peptide
  Xaa (10)..(10): Xaa is HArg
CNTVLCSWAXANSFC

SEQ ID NO: 53 - protein, 15 aa, Artificial
  Synthetic Peptide
  Xaa (10)..(10): Xaa is HArg
CNSVLCSWAXANSFC

SEQ ID NO: 54 - protein, 15 aa, Artificial
  Synthetic Peptide
  Xaa (10)..(10): Xaa is HArg
CNDVLCSWAXANSFC

SEQ ID NO: 55 - protein, 15 aa, Artificial
  Synthetic Peptide
  Xaa (10)..(10): Xaa is HArg
CNEVLCSWAXANSFC

SEQ ID NO: 56 - protein, 15 aa, Artificial
  Synthetic Peptide
  Xaa (10)..(10): Xaa is HArg
  Xaa (11)..(11): Xaa is HyP
CNAVLCSWAXXNSFC

SEQ ID NO: 57 - protein, 15 aa, Artificial
  Synthetic Peptide
  Xaa (10)..(10): Xaa is HArg
CNAVLCSWAXTNSFC

SEQ ID NO: 58 - protein, 15 aa, Artificial
  Synthetic Peptide
  Xaa (10)..(10): Xaa is HArg
CNAVLCSWAXSNSFC

SEQ ID NO: 59 - protein, 12 aa, Artificial
  Synthetic Peptide
CAEIYKCWRGRC

SEQ ID NO: 60 - protein, 12 aa, Artificial
  Synthetic Peptide
CDTLYKCWRGRC

SEQ ID NO: 61 - protein, 12 aa, Artificial
  Synthetic Peptide
CESLYKCWRGRC

SEQ ID NO: 62 - protein, 12 aa, Artificial
  Synthetic Peptide
CNTLYKCWRGKC

SEQ ID NO: 63 - protein, 12 aa, Artificial
  Synthetic Peptide
CTELYKCWRGRC

(19) United States
(12) Patent Application Publication
(10) Pub. No.: US 2023/0183291 A1
(43) Pub. Date: Jun. 15, 2023

(54) ANTI-INFECTIVE BICYCLIC PEPTIDE LIGANDS

(71) Applicant: Bicycle Tx Limited, Cambridge (GB)

(72) Inventors: Nicholas KEEN, Cambridge (GB); Katerine VAN RIETSCHOTEN, Cambridge (GB); Liuhong CHEN, Cambridge (GB); Maximilian HARMAN, Cambridge (GB); Michael SKYNNER, Cambridge (GB); Paul BESWICK, Cambridge (GB); Yuliya DEMYDCHUK, Cambridge (GB)

(21) Appl. No.: 17/924,896

(22) PCT Filed: May 14, 2021
(86) PCT No.: PCT/GB2021/051164

§ 371 Date: Nov. 11, 2022

Related U.S. Application Data
(60) Provisional application No. 63/025,552, filed on May 15, 2020.
(60) Provisional application No. 63/135,213, filed on Jan. 8, 2021.

(51) Int. Cl. C07K 7/08; A61K 38/10

(52) U.S. Cl. CPC C07K 7/08; A61K 38/10

(57) ABSTRACT

The present invention relates to polypeptides which are covalently bound to molecular scaffolds such that two or more peptide loops are subtended between attachment points to the scaffold. In particular, the invention describes peptides which are high affinity binders of ACE2. The invention also includes pharmaceutical compositions comprising said polypeptides and to the use of said polypeptides in suppressing or treating a disease or disorder mediated by ACE2, such as infection of COVID-19 or for providing prophylaxis to a subject at risk of infection of COVID-19.

### FIELD OF THE INVENTION

The present invention relates to polypeptides which are covalently bound to molecular scaffolds such that two or more peptide loops are subtended between attachment points to the scaffold. In particular, the invention describes peptides which are high affinity binders of ACE2. The invention also includes pharmaceutical compositions comprising said polypeptides and to the use of said polypeptides in suppressing or treating a disease or disorder mediated by ACE2, such as infection of COVID-19 or for providing prophylaxis to a subject at risk of infection of COVID-19.

### BACKGROUND OF THE INVENTION

Coronavirus disease 2019 (COVID-19) is an infectious disease caused by severe acute respiratory syndrome coronavirus 2 (SARS-CoV-2). The disease was first identified in December 2019 in Wuhan, the capital of China's Hubei province, and spread globally, resulting in a pandemic. Common symptoms include fever, cough, and shortness of breath. Other symptoms may include fatigue, muscle pain, diarrhea, sore throat, loss of smell, and abdominal pain. The time from exposure to onset of symptoms is typically around five days but may range from two to fourteen days. While the majority of cases result in mild symptoms, some progress to viral pneumonia and multi-organ failure. As of 6 Jan. 2021, more than 86 million cases have been reported globally, resulting in more than 1.8 million deaths.

The virus is primarily spread between people during close contact, often via droplets produced by coughing, sneezing, or talking. While these droplets are produced when breathing out, they usually fall to the ground or onto surfaces rather than being infectious over long distances. People may also become infected by touching a contaminated surface and then their face. The virus can survive on surfaces for up to 72 hours. It is most contagious during the first three days after the onset of symptoms, although spread may be possible before symptoms appear and in later stages of the disease.

Currently, there is no vaccine or specific antiviral treatment for COVID-19. Management involves treatment of symptoms, supportive care, isolation, and experimental measures. The World Health Organization (WHO) declared the 2019-2020 coronavirus outbreak a Public Health Emergency of International Concern (PHEIC) on 30 Jan. 2020 and a pandemic on 11 Mar. 2020. Local transmission of the disease has been recorded in many countries across all six WHO regions.

There is therefore a great need to provide an effective prophylactic and/or therapeutic treatment intended to avoid or ameliorate the symptoms associated with COVID-19 infection.

### SUMMARY OF THE INVENTION

According to a first aspect of the invention, there is provided a peptide ligand specific for ACE2 comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

According to a further aspect of the invention, there is provided a pharmaceutical composition comprising the peptide ligand as defined herein in combination with one or more pharmaceutically acceptable excipients.

According to a further aspect of the invention, there is provided the peptide ligand as defined herein for use in suppressing or treating a disease or disorder mediated by infection of COVID-19 or for providing prophylaxis to a subject at risk of infection of COVID-19.

### DETAILED DESCRIPTION OF THE INVENTION

According to a first aspect of the invention, there is provided a peptide ligand specific for ACE2 comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

References herein to “ACE2” refer to angiotensin-converting enzyme 2 which is an enzyme attached to the outer surface (cell membranes) of cells in the lungs, arteries, heart, kidney, and intestines. ACE2 is known to serve as the entry point into cells for some coronaviruses, such as COVID-19. Without being bound by theory it is believed that the virus that has caused the COVID-19 pandemic (SARS-CoV-2) uses ACE2 (which is bound to the surface of lung airway cells) to enter tissue and cause disease. The same protein ACE2 seems to protect the lung from injury caused by excessive inflammation. It is believed that administration of a peptide ligand which binds to ACE2 could prevent the virus entering cells and prevent the damaging inflammation caused by the virus (which seems to be the major cause of death from this infection).

Thus, the invention finds great utility in the treatment for severe COVID-19 and could even be used to protect people from the current pandemic and any future coronavirus outbreaks.

In one embodiment, said loop sequences comprise 4, 5, 6 or 8 amino acids.

In a further embodiment, said loop sequences comprise 4, 6 or 8 amino acids.

In one embodiment, said loop sequences comprise three reactive groups separated by two loop sequences one of which consists of 4 amino acids and the other of which consists of 8 amino acids.

In a further embodiment, said loop sequences comprise three reactive groups separated by two loop sequences one of which consists of 4 amino acids and the other of which consists of 8 amino acids and the bicyclic peptide ligand comprises an amino acid sequence which is selected from:

(SEQ ID NO: 1)
CᵢHKFPCᵢᵢRDPQQYLFCᵢᵢᵢ;
(SEQ ID NO: 2)
CᵢTSPMCᵢᵢYVLKHQNRCᵢᵢᵢ;
(SEQ ID NO: 3)
CᵢTRPWCᵢᵢHSLLPRATCᵢᵢᵢ;
(SEQ ID NO: 4)
CᵢGRQFCᵢᵢHTLMPRHLCᵢᵢᵢ;
(SEQ ID NO: 5)
CᵢVRSHCᵢᵢSSLLPRIHCᵢᵢᵢ;
(SEQ ID NO: 9)
CᵢAPILCᵢᵢRWAERQGYCᵢᵢᵢ;
(SEQ ID NO: 10)
CᵢNAVLCᵢᵢSWARANSFCᵢᵢᵢ
(herein referred to as BCY17688);
(SEQ ID NO: 11)
CᵢNAVLCᵢᵢS[1Nal]ARANSFCᵢᵢᵢ;
(SEQ ID NO: 12)
CᵢNAVLCᵢᵢS[2Nal]ARANSFCᵢᵢᵢ;
(SEQ ID NO: 13)
CᵢNAVLCᵢᵢSW[Aib]RANSFCᵢᵢᵢ;
(SEQ ID NO: 14)
CᵢNAVLCᵢᵢSWA[HArg]ANSFCᵢᵢᵢ;
(SEQ ID NO: 19)
CᵢNAVLCᵢᵢSWA[Arg(Me)]ANSFCᵢᵢᵢ;
(SEQ ID NO: 20)
CᵢNKVLCᵢᵢSWASRNSMCᵢᵢᵢ;
(SEQ ID NO: 21)
CᵢNQVLCᵢᵢRWAQVNSMCᵢᵢᵢ;
(SEQ ID NO: 22)
CᵢNRVLCᵢᵢAWATANSMCᵢᵢᵢ;
(SEQ ID NO: 23)
CᵢNTVLCᵢᵢNWARYNSLCᵢᵢᵢ;
(SEQ ID NO: 24)
CᵢNTVLCᵢᵢAWARANSYCᵢᵢᵢ;
(SEQ ID NO: 25)
CᵢNTVLCᵢᵢGWARANSLCᵢᵢᵢ;
(SEQ ID NO: 26)
CᵢNTVLCᵢᵢAWAMNNSMCᵢᵢᵢ;
(SEQ ID NO: 27)
CᵢNTVLCᵢᵢAWATQNSLCᵢᵢᵢ;
(SEQ ID NO: 28)
CᵢSPVLCᵢᵢAWATRNSLCᵢᵢᵢ;
(SEQ ID NO: 29)
CᵢNA[tBuGly]LCᵢᵢSWARANSFCᵢᵢᵢ;
(SEQ ID NO: 30)
CᵢNA[tBuAla]LCᵢᵢSWARANSFCᵢᵢᵢ;
(SEQ ID NO: 31)
CᵢNAV[tBuGly]CᵢᵢSWARANSFCᵢᵢᵢ;
(SEQ ID NO: 32)
CᵢNAV[tBuAla]CᵢᵢSWARANSFCᵢᵢᵢ;
(SEQ ID NO: 33)
CᵢNAV[Cba]CᵢᵢSWARANSFCᵢᵢᵢ;
(SEQ ID NO: 34)
CᵢNAVLCᵢᵢSWARANS[2MePhe]Cᵢᵢᵢ;
(SEQ ID NO: 35)
CᵢNAVLCᵢᵢSWARANS[SMePhe]Cᵢᵢᵢ;
(SEQ ID NO: 36)
CᵢNAVLCᵢᵢSWARANS[MePhe]Cᵢᵢᵢ;
(SEQ ID NO: 37)
CᵢNAVLCᵢᵢSWARANS[2ClPhelCᵢᵢᵢ;
(SEQ ID NO: 38)
CᵢNAVLCᵢᵢSWARANS[3ClPhelCᵢᵢᵢ;
(SEQ ID NO: 39)
CᵢNAVLCᵢᵢSWARANS[4ClPhe]Cᵢᵢᵢ;
(SEQ ID NO: 40)
CᵢNAVLCᵢᵢSWARANS[2FPhe]Cᵢᵢᵢ;
(SEQ ID NO: 41)
CᵢNAVLCᵢᵢSWARANS[3FPhe]Cᵢᵢᵢ;
(SEQ ID NO: 42)
CᵢNAVLCᵢᵢSWA[Agb]ANSFCᵢᵢᵢ;
(SEQ ID NO: 43)
CᵢNAVLCᵢᵢSWARANSYCᵢᵢᵢ;
(SEQ ID NO: 44)
CᵢN[HyP]VLCᵢᵢSWA[HArg]ANSFCᵢᵢᵢ;
(SEQ ID NO: 45)
CᵢNAVLCᵢᵢSWA[HArg][dA]NSFCᵢᵢᵢ;
(SEQ ID NO: 46)
CᵢN[dD]VLCᵢᵢSWA[HArg]ANSFCᵢᵢᵢ;
(SEQ ID NO: 47)
CᵢN[dA]VLCᵢᵢSWA[HArg]ANSFCᵢᵢᵢ;
(SEQ ID NO: 48)
CᵢNRVLCᵢᵢAWATANS[Hse(Me)]Cᵢᵢᵢ;
(SEQ ID NO: 49)
CᵢN[HArg]VLCᵢᵢSWA[HArg]ANSFCᵢᵢᵢ;
(SEQ ID NO: 50)
CᵢNTVLCᵢᵢGWA[HArg]ANSLCᵢᵢᵢ;
(SEQ ID NO: 51)
CᵢNTVLCᵢᵢGWARANSLCᵢᵢᵢ;
(SEQ ID NO: 52)
CᵢNTVLCᵢᵢSWA[HArg]ANSFCᵢᵢᵢ;
(SEQ ID NO: 53)
CᵢNSVLCᵢᵢSWA[HArg]ANSFCᵢᵢᵢ;
(SEQ ID NO: 54)
CᵢNDVLCᵢᵢSWA[HArg]ANSFCᵢᵢᵢ;
(SEQ ID NO: 55)
CᵢNEVLCᵢᵢSWA[HArg]ANSFCᵢᵢᵢ;
(SEQ ID NO: 56)
CᵢNAVLCᵢᵢSWA[HArg][HyP]NSFCᵢᵢᵢ;
(SEQ ID NO: 57)
CᵢNAVLCᵢᵢSWA[HArg]TNSFCᵢᵢᵢ;
and
(SEQ ID NO: 58)
CᵢNAVLCᵢᵢSWA[HArg]SNSFCᵢᵢᵢ;

wherein Cᵢ, Cᵢᵢ and Cᵢᵢᵢ represent first, second and third cysteine residues, respectively, 1Nal represents 1-naphthylalanine, 2Nal represents 2-naphthylalanine, Aib represents aminoisobutyric acid, HArg represents homoarginine, Arg(Me) represents δ-N methyl arginine, tBuGly represents t-butyl-glycine, tBuAla represents t-butyl-alanine, Cba represents β-cyclobutylalanine, 2MePhe represents 2-methyl-phenylalanine, 3MePhe represents 3-methyl-phenylalanine, 4MePhe represents 4-methyl-phenylalanine, 2ClPhe represents 2-chloro-phenylalanine, 3ClPhe represents 3-chloro-phenylalanine, 4ClPhe represents 4-chloro-phenylalanine, 2FPhe represents 2-fluoro-phenylalanine, 3FPhe represents 3-fluoro-phenylalanine, Agb represents 2-amino-4-guanidinobutyric acid, HyP represents hydroxyproline and Hse(Me) represents homoserine-methyl, or a pharmaceutically acceptable salt thereof.

In a further embodiment, said loop sequences comprise three reactive groups separated by two loop sequences one of which consists of 4 amino acids and the other of which consists of 8 amino acids and the bicyclic peptide ligand comprises an amino acid sequence which is selected from:

(SEQ ID NO: 1)
CᵢHKFPCᵢᵢRDPQQYLFCᵢᵢᵢ;
(SEQ ID NO: 2)
CᵢTSPMCᵢᵢYVLKHQNRCᵢᵢᵢ;
(SEQ ID NO: 3)
CᵢTRPWCᵢᵢHSLLPRATCᵢᵢᵢ;
(SEQ ID NO: 4)
CᵢGRQFCᵢᵢHTLMPRHLCᵢᵢᵢ;
(SEQ ID NO: 5)
CᵢVRSHCᵢᵢSSLLPRIHCᵢᵢᵢ;
(SEQ ID NO: 9)
CᵢAPILCᵢᵢRWAERQGYCᵢᵢᵢ;
(SEQ ID NO: 10)
CᵢNAVLCᵢᵢSWARANSFCᵢᵢᵢ
(SEQ ID NO: 11)
CᵢNAVLCᵢᵢS[1Nal]ARANSFCᵢᵢᵢ;
(SEQ ID NO: 12)
CᵢNAVLCᵢᵢS[2Nal]ARANSFCᵢᵢᵢ;
(SEQ ID NO: 13)
CᵢNAVLCᵢᵢSW[Aib]RANSFCᵢᵢᵢ;
and
(SEQ ID NO: 14)
CᵢNAVLCᵢᵢSWA[HArg]ANSFCᵢᵢᵢ;

wherein Cᵢ, Cᵢᵢ and Cᵢᵢᵢ represent first, second and third cysteine residues, respectively, 1Nal represents 1-naphthylalanine, 2Nal represents 2-naphthylalanine, Aib represents aminoisobutyric acid, HArg represents homoarginine, or a pharmaceutically acceptable salt thereof.

In a yet further embodiment, said loop sequences comprise three reactive groups separated by two loop sequences one of which consists of 4 amino acids and the other of which consists of 8 amino acids and the bicyclic peptide ligand comprises an amino acid sequence which is selected from:

(SEQ ID NO: 1)
CᵢHKFPCᵢᵢRDPQQYLFCᵢᵢᵢ;
(SEQ ID NO: 2)
CᵢTSPMCᵢᵢYVLKHQNRCᵢᵢᵢ;
(SEQ ID NO: 3)
CᵢTRPWCᵢᵢHSLLPRATCᵢᵢᵢ;
(SEQ ID NO: 4)
CᵢGRQFCᵢᵢHTLMPRHLCᵢᵢᵢ;
and
(SEQ ID NO: 5)
CᵢVRSHCᵢᵢSSLLPRIHCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 4 amino acids and the other of which consists of 8 amino acids, the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15296);
A-(SEQ ID NO: 2)-A (herein referred to as BCY15295);
A-(SEQ ID NO: 3)-A (herein referred to as BCY15293);
A-(SEQ ID NO: 4)-A (herein referred to as BCY15292);
A-(SEQ ID NO: 5)-A (herein referred to as BCY15291);
A-(SEQ ID NO: 9)-A (herein referred to as BCY15425);
A-(SEQ ID NO: 10)-A (herein referred to as BCY15429);
A-(SEQ ID NO: 10)-A-[K(PYA)] (herein referred to as BCY17344);
Ac-(SEQ ID NO: 10) (herein referred to as BCY17663);
Ac-A-(SEQ ID NO: 10)-A (herein referred to as BCY17691);
Ac-(SEQ ID NO: 14) (herein referred to as BCY18259);
Ac-(SEQ ID NO: 14)-[K(PYA)] (herein referred to as BCY18784);
A-(SEQ ID NO: 20)-A (herein referred to as BCY17157);
A-(SEQ ID NO: 21)-A (herein referred to as BCY17158);
A-(SEQ ID NO: 22)-A (herein referred to as BCY17159);
A-(SEQ ID NO: 23)-A (herein referred to as BCY17160);
A-(SEQ ID NO: 24)-A (herein referred to as BCY17161);
A-(SEQ ID NO: 25)-A (herein referred to as BCY17162);
A-(SEQ ID NO: 26)-A (herein referred to as BCY17163);
A-(SEQ ID NO: 27)-A (herein referred to as BCY17164);
A-(SEQ ID NO: 28)-A (herein referred to as BCY17165);
A-(SEQ ID NO: 29)-A (herein referred to as BCY17330);
A-(SEQ ID NO: 30)-A (herein referred to as BCY17331);
A-(SEQ ID NO: 31)-A (herein referred to as BCY17332);
A-(SEQ ID NO: 32)-A (herein referred to as BCY17333);
A-(SEQ ID NO: 33)-A (herein referred to as BCY17334);
A-(SEQ ID NO: 34)-A (herein referred to as BCY17335);
A-(SEQ ID NO: 35)-A (herein referred to as BCY17336);
A-(SEQ ID NO: 36)-A (herein referred to as BCY17337);
A-(SEQ ID NO: 37)-A (herein referred to as BCY17338);
A-(SEQ ID NO: 38)-A (herein referred to as BCY17339);
A-(SEQ ID NO: 39)-A (herein referred to as BCY17340);
A-(SEQ ID NO: 40)-A (herein referred to as BCY17341);
A-(SEQ ID NO: 41)-A (herein referred to as BCY17342);
A-(SEQ ID NO: 42)-A (herein referred to as BCY17689);
A-(SEQ ID NO: 43)-A (herein referred to as BCY17690);
Ac-(SEQ ID NO: 44) (herein referred to as BCY18210);
Ac-(SEQ ID NO: 45) (herein referred to as BCY18256);
Ac-(SEQ ID NO: 46) (herein referred to as BCY18257);
Ac-(SEQ ID NO: 47) (herein referred to as BCY18258);
Ac-(SEQ ID NO: 48)-[K(PYA)] (herein referred to as BCY18349);
Ac-(SEQ ID NO: 49) (herein referred to as BCY18350);
Ac-(SEQ ID NO: 50)-A-[K(PYA)] (herein referred to as BCY18549);
Ac-(SEQ ID NO: 50)-[K(PYA)] (herein referred to as BCY18551);
Ac-(SEQ ID NO: 51)-[K(PYA)] (herein referred to as BCY18550);
Ac-(SEQ ID NO: 52) (herein referred to as BCY18657);
Ac-(SEQ ID NO: 53) (herein referred to as BCY18658);
Ac-(SEQ ID NO: 54) (herein referred to as BCY18659);
Ac-(SEQ ID NO: 55) (herein referred to as BCY18783);
Ac-(SEQ ID NO: 56) (herein referred to as BCY19024);
Ac-(SEQ ID NO: 57) (herein referred to as BCY19025); and
Ac-(SEQ ID NO: 58) (herein referred to as BCY19026);

wherein PYA represents propargyl-acid.

In a yet further embodiment, said loop sequences comprise three reactive groups separated by two loop sequences one of which consists of 4 amino acids and the other of which consists of 8 amino acids, the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15296);
A-(SEQ ID NO: 2)-A (herein referred to as BCY15295);
A-(SEQ ID NO: 3)-A (herein referred to as BCY15293);
A-(SEQ ID NO: 4)-A (herein referred to as BCY15292);
A-(SEQ ID NO: 5)-A (herein referred to as BCY15291);
A-(SEQ ID NO: 9)-A (herein referred to as BCY15425); and
A-(SEQ ID NO: 10)-A (herein referred to as BCY15429).

In a yet further embodiment, said loop sequences comprise three reactive groups separated by two loop sequences one of which consists of 4 amino acids and the other of which consists of 8 amino acids, the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15296);
A-(SEQ ID NO: 2)-A (herein referred to as BCY15295);
A-(SEQ ID NO: 3)-A (herein referred to as BCY15293);
A-(SEQ ID NO: 4)-A (herein referred to as BCY15292); and
A-(SEQ ID NO: 5)-A (herein referred to as BCY15291).

In a still yet further embodiment, said loop sequences comprise three reactive groups separated by two loop sequences one of which consists of 4 amino acids and the other of which consists of 8 amino acids, the molecular scaffold is TATA, the bicyclic peptide additionally comprises N- and/or C-terminal additions and a labelling moiety, such as fluorescein (FI), and comprises an amino acid sequence which is selected from:
A-(SEQ ID NO: 1)-A-[Sar₆]-[KFI] (herein referred to as BCY15288);
A-(SEQ ID NO: 2)-A-[Sar₆]-[KFI] (herein referred to as BCY15287);
A-(SEQ ID NO: 3)-A-[Sar₆]-[KFI] (herein referred to as BCY15285);
A-(SEQ ID NO: 4)-A-[Sar₆]-[KFI] (herein referred to as BCY15284);
A-(SEQ ID NO: 5)-A-[Sar₆]-[KFI] (herein referred to as BCY15283);
A-(SEQ ID NO: 9)-A-[Sar₆]-[KFI] (herein referred to as BCY15419);
A-(SEQ ID NO: 10)-A-[Sar₆]-[KFI] (herein referred to as BCY15423);
A-(SEQ ID NO: 11)-A-[Sar₆]-[KFI] (herein referred to as BCY16866);
A-(SEQ ID NO: 12)-A-[Sar₆]-[KFI] (herein referred to as BCY16867);
A-(SEQ ID NO: 13)-A-[Sar₆]-[KFI] (herein referred to as BCY16872);
A-(SEQ ID NO: 14)-A-[Sar₆]-[KFI] (herein referred to as BCY16874); and
A-(SEQ ID NO: 19)-A-[Sar₆]-[KFI] (herein referred to as BCY16863).

In a still yet further embodiment, said loop sequences comprise three reactive groups separated by two loop sequences one of which consists of 4 amino acids and the other of which consists of 8 amino acids, the molecular scaffold is TATA, the bicyclic peptide additionally comprises N- and/or C-terminal additions and a labelling moiety, such as fluorescein (FI), and comprises an amino acid sequence which is selected from:
A-(SEQ ID NO: 1)-A-[Sar₆]-[KFI] (herein referred to as BCY15288);
A-(SEQ ID NO: 2)-A-[Sar₆]-[KFI] (herein referred to as BCY15287);
A-(SEQ ID NO: 3)-A-[Sar₆]-[KFI] (herein referred to as BCY15285);
A-(SEQ ID NO: 4)-A-[Sar₆]-[KFI] (herein referred to as BCY15284); and
A-(SEQ ID NO: 5)-A-[Sar₆]-[KFI] (herein referred to as BCY15283).

In an alternative embodiment, said loop sequences comprise three reactive groups separated by two loop sequences one of which consists of 5 amino acids and the other of which consists of 4 amino acids.

In a further embodiment, said loop sequences comprise three reactive groups separated by two loop sequences one of which consists of 5 amino acids and the other of which consists of 4 amino acids and the bicyclic peptide ligand comprises an amino acid sequence which is selected from:

(SEQ ID NO: 15)
CᵢLELYQCᵢWRGKCᵢᵢ;
(SEQ ID NO: 16)
CᵢPSQYKCᵢᵢWRGKCᵢᵢᵢ;
(SEQ ID NO: 17)
CᵢLEVYKCᵢᵢWRGKCᵢᵢᵢ;
(SEQ ID NO: 59)
CᵢAEIYKCᵢᵢWRGRCᵢᵢᵢ;
(SEQ ID NO: 60)
CᵢDTLYKCᵢᵢWRGRCᵢᵢᵢ;
(SEQ ID NO: 61)
CᵢESLYKCᵢᵢWRGRCᵢᵢᵢ;
(SEQ ID NO: 62)
CᵢNTLYKCᵢᵢWRGKCᵢᵢᵢ;
and
(SEQ ID NO: 63)
CᵢTELYKCᵢᵢWRGRCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 5 amino acids and the other of which consists of 4 amino acids and the bicyclic peptide ligand comprises an amino acid sequence which is selected from:

(SEQ ID NO: 15)
CᵢLELYQCᵢWRGKCᵢᵢ;
(SEQ ID NO: 16)
CᵢPSQYKCᵢᵢWRGKCᵢᵢᵢ;
and
(SEQ ID NO: 17)
CᵢLEVYKCᵢᵢWRGKCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 5 amino acids and the other of which consists of 4 amino acids, the molecular scaffold is TATA, the bicyclic peptide ligand additionally comprises N- and/or C-terminal additions and comprises an amino acid sequence which is selected from:
A-(SEQ ID NO: 15)-A (herein referred to as BCY15426);
A-(SEQ ID NO: 16)-A (herein referred to as BCY15427);
A-(SEQ ID NO: 17)-A (herein referred to as BCY15428);
A-(SEQ ID NO: 59)-A (herein referred to as BCY17152);
A-(SEQ ID NO: 60)-A (herein referred to as BCY17153);
A-(SEQ ID NO: 61)-A (herein referred to as BCY17154);
A-(SEQ ID NO: 62)-A (herein referred to as BCY17155); and
A-(SEQ ID NO: 63)-A (herein referred to as BCY17156).

In a yet further embodiment, said loop sequences comprise three reactive groups separated by two loop sequences one of which consists of 5 amino acids and the other of which consists of 4 amino acids, the molecular scaffold is TATA, the bicyclic peptide ligand additionally comprises N- and/or C-terminal additions and comprises an amino acid sequence which is selected from:
A-(SEQ ID NO: 15)-A (herein referred to as BCY15426);
A-(SEQ ID NO: 16)-A (herein referred to as BCY15427); and
A-(SEQ ID NO: 17)-A (herein referred to as BCY15428).

In a still yet further embodiment, said loop sequences comprise three reactive groups separated by two loop sequences one of which consists of 5 amino acids and the other of which consists of 4 amino acids, the molecular scaffold is TATA, the bicyclic peptide additionally comprises N- and/or C-terminal additions and a labelling moiety, such as fluorescein (FI), and comprises an amino acid sequence which is:
A-(SEQ ID NO: 15)-A-[Sar₆]-[KFI] (herein referred to as BCY15420);
A-(SEQ ID NO: 16)-A-[Sar₆]-[KFI] (herein referred to as BCY15421); and
A-(SEQ ID NO: 17)-A-[Sar₆]-[KFI] (herein referred to as BCY15422).

In an alternative embodiment, said loop sequences comprise three reactive groups separated by two loop sequences one of which consists of 5 amino acids and the other of which consists of 8 amino acids.

In a further embodiment, said loop sequences comprise three reactive groups separated by two loop sequences one of which consists of 5 amino acids and the other of which consists of 8 amino acids and the bicyclic peptide ligand comprises an amino acid sequence which is:

(SEQ ID NO: 18)
CᵢAN[Aᵢb]VLCᵢᵢSWARANSFCᵢᵢᵢ;

wherein Cᵢ, Cᵢᵢ and Cᵢᵢᵢ represent first, second and third cysteine residues, respectively, Aib represents aminoisobutyric acid, or a pharmaceutically acceptable salt thereof.

In a yet further embodiment, said loop sequences comprise three reactive groups separated by two loop sequences one of which consists of 5 amino acids and the other of which consists of 4 amino acids, the molecular scaffold is TATA, the bicyclic peptide ligand additionally comprises N- and/or C-terminal additions and a labelling moiety, such as fluorescein (FI), and comprises an amino acid sequence which is:
A-(SEQ ID NO: 18)-A-[Sar₆]-[KFI] (herein referred to as BCY16871).

In an alternative embodiment, said loop sequences comprise three reactive groups separated by two loop sequences one of which consists of 6 amino acids and the other of which consists of 4 amino acids.

In a further embodiment, said loop sequences comprise three reactive groups separated by two loop sequences one of which consists of 6 amino acids and the other of which consists of 4 amino acids and the bicyclic peptide ligand comprises an amino acid sequence which is selected from:

(SEQ ID NO: 6)
CᵢGREELPCᵢᵢRIKLCᵢᵢᵢ;
and
(SEQ ID NO: 7)
CᵢLRSYNLCᵢᵢPRINCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 6 amino acids and the other of which consists of 4 amino acids, the molecular scaffold is TATA, the bicyclic peptide ligand additionally comprises N- and/or C-terminal additions and comprises an amino acid sequence which is
A-(SEQ ID NO: 6)-A (herein referred to as BCY15298); and
A-(SEQ ID NO: 7)-A (herein referred to as BCY15294).

In a still yet further embodiment, said loop sequences comprise three reactive groups separated by two loop sequences one of which consists of 6 amino acids and the other of which consists of 4 amino acids, the molecular scaffold is TATA, the bicyclic peptide additionally comprises N- and/or C-terminal additions and a labelling moiety, such as fluorescein (FI), and comprises an amino acid sequence which is selected from:
A-(SEQ ID NO: 6)-A-[Sar₆]-[KFI] (herein referred to as BCY15290); and
A-(SEQ ID NO: 7)-A-[Sar₆]-[KFI] (herein referred to as BCY15286).

In an alternative embodiment, said loop sequences comprise three reactive groups separated by two loop sequences both of which consist of 6 amino acids.

In a further embodiment, said loop sequences comprise three reactive groups separated by two loop sequences both of which consist of 6 amino acids and the bicyclic peptide ligand comprises an amino acid sequence which is:

(SEQ ID NO: 8)
CᵢHRDFPRCᵢᵢTWETQWCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences both of which consist of 6 amino acids, the molecular scaffold is TATA and the bicyclic peptide ligand additionally comprises N- and/or C-terminal additions and comprises an amino acid sequence which is:
A-(SEQ ID NO: 8)-A (herein referred to as BCY15297).

In a still yet further embodiment, said loop sequences comprise three reactive groups separated by two loop sequences both of which consist of 6 amino acids, the molecular scaffold is TATA, the bicyclic peptide additionally comprises N- and/or C-terminal additions and a labelling moiety, such as fluorescein (FI), and comprises an amino acid sequence which is:
A-(SEQ ID NO: 8)-A-[Sar₆]-[KFI] (herein referred to as BCY15289).

In one embodiment, the bicyclic peptide of the invention binds to the active site of ACE2. Examples of such active site binding bicyclic peptides include BCY15291, BCY15292, BCY15293 and BCY15296. Without being bound by theory, it is believed that the bicyclic peptides of the invention which bind to the active site of ACE2 are likely to have beneficial physiological effects, such as blood pressure alteration (see FIG. 2 of Verdecchia et al (2020) European Journal of Internal Medicine 76, 14-20).

In an alternative embodiment, the bicyclic peptide of the invention binds to an epitope of ACE2 which is other than the active site. Examples of such non-active site binding bicyclic peptides include BCY15294, BCY15295, BCY15297, BCY15298, BCY15425, BCY15426, BCY15427, BCY15428, BCY15423, BCY16871, BCY16866, BCY16867, BCY16872 and BCY16874. Without being bound by theory, it is believed that the bicyclic peptides of the invention which bind to an epitope of ACE2 which is other than the active site of ACE2 are likely to have beneficial properties by blocking viral entry without demonstrating other effects (see Verdecchia et al (2020) European Journal of Internal Medicine 76, 14-20).

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

Nomenclature

Numbering

When referring to amino acid residue positions within peptides of the invention, cysteine residues (Cᵢ, Cᵢᵢ and Cᵢᵢᵢ) are omitted from the numbering as they are invariant, therefore, the numbering of amino acid residues within peptides of the invention is referred to as below:

(SEQ ID NO: 1)
Cᵢ-H₁-K₂-F₃-P₄-Cᵢᵢ-R₅-D₆-P₇-Q₈-Q₉-Y₁₀-L₁₁-F₁₂-Cᵢᵢᵢ

For the purpose of this description, all bicyclic peptides are assumed to be cyclised with TATA and yielding a tri-substituted structure. Cyclisation with TATA occurs on the first, second and third reactive groups (i.e. Cᵢ, Cᵢᵢ, Cᵢᵢᵢ).

Molecular Format

N- or C-terminal extensions to the bicycle core sequence are added to the left or right side of the sequence, separated by a hyphen. For example, an N-terminal βAla-Sar10-Ala tail would be denoted as:

3Ala-Sar10-A-(SEQ ID NO: X).

Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. Certain ligands demonstrate cross-reactivity across Lipid II from different bacterial species and hence are able to treat infections caused by multiple species of bacteria. Other ligands may be highly specific for the Lipid II of certain bacterial species which may be advantageous for treating an infection without collateral damage to the beneficial flora of the patient;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity.

Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Cα-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I, and ¹³¹I nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

Molecular Scaffold

Molecular scaffolds are described in, for example, WO 2009/098450 and references cited therein, particularly WO 2004/077062 and WO 2006/078161.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene or iodoacetamide. Other scaffold reactive groups that are used to selectively couple compounds to cysteines in proteins are maleimides, αβ unsaturated carbonyl containing compounds and α-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene.

In one embodiment, the molecular scaffold is 1,1′,1″-(1,3,5-triazinane-1,3,5-triyl)triprop-2-en-1-one (also known as triacryloylhexahydro-s-triazine (TATA):

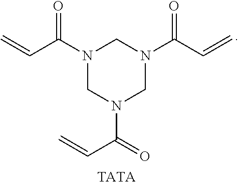

Thus, following cyclisation with the bicyclic peptides of the invention on the Cᵢ, Cᵢᵢ, and Cᵢᵢᵢ cysteine residues, the molecular scaffold forms a tri-substituted 1,1′,1″-(1,3,5-triazinane-1,3,5-triyl)tripropan-1-one derivative of TATA having the following structure:

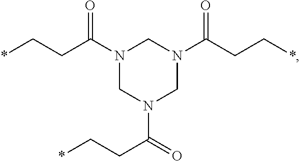

wherein * denotes the point of attachment of the three cysteine residues.

Reactive Groups

The molecular scaffold of the invention may be bonded to the polypeptide via functional or reactive groups on the polypeptide. These are typically formed from the side chains of particular amino acids found in the polypeptide polymer. Such reactive groups may be a cysteine side chain, a [Dap(Me)] group, a lysine side chain, or an N-terminal amine group or any other suitable reactive group. Details may be found in WO 2009/098450. In one embodiment, the reactive groups are all cysteine residues.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment of the invention, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

In one embodiment, an polypeptide with three reactive groups has the sequence (X)ₗY(X)ₘY(X)ₙY(X)ₒ, wherein Y represents an amino acid with a reactive group, X represents a random amino acid, m and n are numbers between 3 and 6 defining the length of intervening polypeptide segments, which may be the same or different, and I and o are numbers between 0 and 20 defining the length of flanking polypeptide segments.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis, et al., Nat Chem Biol 2009, 5 (7), 502-7.

Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al. (supra).

Thus, the invention also relates to manufacture of polypeptides selected as set out herein, wherein the manufacture comprises optional further steps as explained below. In one embodiment, these steps are carried out on the end product polypeptide made by chemical synthesis.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively, additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compounds of the invention can be used alone or in combination with another agent or agents.

The compounds of the invention can also be used in combination with biological therapies such as nucleic acid based therapies, antibodies, bacteriophage or phage lysins.

The route of administration of pharmaceutical compositions according to the invention may be any of those commonly known to those of ordinary skill in the art. For therapy, the peptide ligands of the invention can be administered to any patient in accordance with standard techniques. Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. Preferably, the pharmaceutical compositions according to the invention will be administered parenterally. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 10 μg to 250 mg of selected peptide ligand per kilogram of body weight, with doses of between 100 μg to 25 mg/kg/dose being more commonly used.

A composition containing a peptide ligand according to the present invention may be utilised in therapeutic settings to treat a microbial infection or to provide prophylaxis to a subject at risk of infection e.g. undergoing surgery, chemotherapy, artificial ventilation or other condition or planned intervention. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

Therapeutic Uses

The bicyclic peptides of the invention have specific utility as ACE2 binding agents.

It will be appreciated that the present invention may be useful as a prophylactic or therapeutic agent for the treatment of any suitable respiratory disorder.

Thus, according to a further aspect of the invention there is provided a peptide ligand as defined herein for use in the prophylaxis or treatment of a respiratory disorder.

According to a further aspect of the invention, there is provided a method of suppressing or treating a respiratory disorder, which comprises administering to a patient in need thereof the peptide ligand as defined herein.

The invention finds particular utility in the prophylaxis or treatment of a respiratory disorder which is mediated by an inflammatory response within the lung. It will be appreciated that such inflammatory responses may be mediated by either a bacterial infection or a viral infection.

In one embodiment, the inflammatory response is mediated by a viral infection.

In a further embodiment, the viral infection is an infection of: rhinovirus; respiratory syncytial virus (RSV); human metapneumovirus (hMPV); influenza; severe acute respiratory syndrome coronavirus (SARS-CoV or SARS-CoV-1); severe acute respiratory syndrome-related coronavirus (SARSr-CoV); severe acute respiratory syndrome coronavirus 2 (SARS-CoV-2); or Middle East respiratory syndrome coronavirus (MERS-CoV).

In a yet further embodiment the viral infection is an infection of severe acute respiratory syndrome coronavirus 2 (SARS-CoV-2).

It will therefore be appreciated that the respiratory disorders intended to be alleviated or treated by the pharmaceutical composition of the invention includes those caused by the above mentioned viruses. Thus, in one embodiment, the respiratory disorder is selected from: Coronavirus disease 2019 (COVID-19), severe acute respiratory syndrome (SARS), Middle East respiratory syndrome (MERS), acute lung injury (ALI), acute respiratory distress syndrome (ARDS) and pulmonary arterial hypertension (PAH).

In a further embodiment, the respiratory disorder is Coronavirus disease 2019 (COVID-19).

Polypeptide ligands selected according to the method of the present invention may be employed in in vivo therapeutic applications, in vitro and in vivo diagnostic applications, in vitro assay and reagent applications, and the like. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

References herein to the term “suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available.

The invention is further described below with reference to the following examples.

### EXAMPLES

Materials and Methods

Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology.

Alternatively, peptides were purified using HPLC and following isolation they were modified with the required molecular scaffold (namely, TATA). For this, linear peptide was diluted with 50:50 MeCN:H₂O up to ˜35 mL, ˜500 μL of 100 mM scaffold in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA.

Following lyophilisation, the modified peptide was purified as above, while replacing the Luna C8 with a Gemini C18 column (Phenomenex), and changing the acid to 0.1% trifluoroacetic acid. Pure fractions containing the correct scaffold-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

Biological Data

1. Affinity Determination by Fluorescence Polarization (FP) Direct Binding

Bicyclic peptides labelled with fluorescein (tracers) were screened in a fluorescence polarisation direct binding assay to determine affinity (Kd) for variants of the ACE2 protein. Tracers were added at 1 nM final to a titration of individual ACE2 Spike Protein variants in assay buffer (PBS+0.01% Tween20, pH7.4) to a maximum of 2.54 μM. Fluorescence was measured at 485/520/520 on a BMG PHERAstar FSX plate reader. Where appropriate, ACE2 protein variants alone parallel and perpendicular intensities were subtracted before mP was calculated. Subsequently, mP data was fit to non-linear regression analysis in Dotmatics to generate a Kd value. Where no significant assay window was generated, data was reported to show no binding at maximum concentration of protein. Where a Kd was generated above the top concentration of protein tested, the result was flagged as Kd greater than maximum concentration of protein tested—results associated with this flag may be displayed as Kd>x μM.

Selected bicyclic peptides of the invention were tested in the above mentioned direct binding assay and the results are shown in Table 1:

TABLE 1
Direct Binding Assay Results for Selected
Bicyclic Peptides of the Invention

| Bicyclic Peptide | Kd (μM) |
|---|---|
| BCY15283 | 0.02185 |
| BCY15284 | 0.06547 |
| BCY15285 | 0.1572 |
| BCY15288 | 0.2254 |
| BCY15419 | 0.02037 |
| BCY15420 | 0.0983 |
| BCY15421 | 0.1339 |
| BCY15422 | 0.1299 |
| BCY15423 | 0.00553 |
| BCY16871 | 0.0058 |
| BCY16866 | 0.0026 |
| BCY16867 | 0.0507 |
| BCY16872 | 0.0029 |
| BCY16874 | 0.062 |

2. Affinity Determination by Surface Plasmon Resonance (SPR) Via Single Cycle Kinetics (SCK)

Binding of bicyclic peptides of the invention to Biotinylated Human ACE2, His, Avitag™ protein (ACROBiosystems, AC2-H82E6) was assessed by a SCK analysis. Experiments were performed at 25° C. on a Biacore T200 (Cytiva) running Biacore T200 Control software V2.0.1 and Evaluation software V3.0 (Cytiva). HBS-EP+ (Cytiva) was used as running buffer as well as for ligand and analyte dilutions. Biotin CAPture reagent was loaded onto a Series S Sensor CAP Chip (Cytiva) followed by loading of the ACE2 protein at a flow rate of 2 μl/min. The surface was then allowed to stabilise.

SCK data was obtained using bicyclic peptides of the invention as analytes injected at a flow rate of 30 μl/min to minimise any potential mass transfer effects. A minimum four point 2-fold dilution of analyte based on the affinity of the bicyclic peptides of the invention in running buffer was used without regeneration between each concentration. The association phases were monitored for 100 seconds for each of the four injections of increasing concentrations of analyte and a single dissociation phase was measured for 400 seconds following the last injection of analyte. Regeneration of the sensor chip surface was conducted using the CAP Chip standard regeneration buffer (Cytiva).

The signal from the reference channel F_{c}1 (no ACE2 captured) was subtracted from that of F_{c}2, F_{c}3 and F_{c}4 to correct for bulk effect and differences in non-specific binding to a reference surface. The signal from each blank run (F_{c}2-ACE2 captured but no antigen) was subtracted to correct for differences in surface stability. The double referenced sensorgrams were fitted with the Langmuir (1:1) binding model (Equation 1a below) where the closeness of fit of the data to the model is evaluated using the Chi square value which describes the deviation between the experimental and fitted (observed and expected) curves (Equation 1b below).

$$A + B {{⇌}_{{k}_{d}}}^{{k}_{a}} \begin{matrix}AB & {K}_{D} = \frac{{k}_{d}}{{k}_{a}} & \begin{matrix}{k}_{a} = association rate constant ( {M}^{- 1} {s}^{- 1} ) \\ {k}_{d} = di ssociation rate constant ( {s}^{- 1} )\end{matrix}\end{matrix} \qquad a )$$

$$Chi square = \begin{matrix}\frac{\sum {( {r}_{f} - {r}_{x} )}^{2}}{n - p} & \begin{matrix}rf = fitted value at a given point \\ rx = experimental value at the same point \\ n = number of data points \\ p = number of fitted paraments\end{matrix}\end{matrix} \qquad b )$$

TABLE 2
SPR SCK Direct Binding Assay Results for Selected
Bicyclic Peptides of the Invention

| BCY Number | Geometric Mean K_{D} (nM) |
|---|---|
| BCY15423 | 3.4 |
| BCY15427 | 149.7 |
| BCY15429 | 3.4 |
| BCY16863 | 3.5 |
| BCY16866 | 2.1 |
| BCY16871 | 7.7 |
| BCY16872 | 8.9 |
| BCY16874 | 4.1 |
| BCY17152 | 119.5 |
| BCY17153 | 91.7 |
| BCY17154 | 104.4 |
| BCY17155 | 107.3 |
| BCY17156 | 150.6 |
| BCY17157 | 11.8 |
| BCY17158 | 4.0 |
| BCY17159 | 4.7 |
| BCY17160 | 4.2 |
| BCY17161 | 4.2 |
| BCY17162 | 5.7 |
| BCY17163 | 36.8 |
| BCY17164 | 14.8 |
| BCY17165 | 12.5 |
| BCY17330 | ** |
| BCY17331 | ** |
| BCY17332 | ** |
| BCY17333 | 5.0 |
| BCY17334 | ** |
| BCY17335 | 2.5 |
| BCY17336 | 8.3 |
| BCY17337 | 5.0 |
| BCY17338 | 3.2 |
| BCY17339 | 4.7 |
| BCY17340 | 16.9† |
| BCY17341 | 3.1 |
| BCY17342 | 3.3 |
| BCY17344 | 3.2† |
| BCY17663 | 5.3 |
| BCY17688 | 2.7 |
| BCY17689 | 4.7 |
| BCY17690 | 4.8 |
| BCY17691 | 3.9 |
| BCY18210 | 21.1* |
| BCY18256 | No sig. binding at top conc. tested |
| BCY18257 | No sig. binding at top conc. tested |
| BCY18258 | No sig. binding at top conc. tested |
| BCY18259 | 23.0* |
| BCY18349 | 59.6* |
| BCY18350 | 8.3 |
| BCY18549 | 63.9* |
| BCY18550 | 9.7* |
| BCY18551 | 15.4* |
| BCY18657 | 69.3 |
| BCY18658 | 6.7 |
| BCY18659 | No sig. binding at top conc. tested |
| BCY18783 | No sig. binding at top conc. tested |
| BCY18784 | 25.3 |

*Rₘₐₓ set to 10
**1:1 model unable to fit sensorgrams (fitted curve not reliable)
†High bulk contribution observed-data should be treated with a degree of caution

3. Affinity Determination by Surface Plasmon Resonance (SPR) Via Multiple Cycle Kinetics (MCK)

Binding of bicyclic peptides of the invention to Biotinylated Human ACE2, His, Avitag™ protein (ACROBiosystems, AC2-H82E6) was assessed by a MCK analysis. Experiments were performed at 25° C. on a Biacore T200 (Cytiva) running Biacore T200 Control software V2.0.1 and Evaluation software V3.0 (Cytiva). HBS-EP+(Cytiva) was used as running buffer as well as for ligand and analyte dilutions. Biotin CAPture reagent was loaded onto a Series S Sensor CAP Chip (Cytiva) followed by loading of biotinylated ACE2 at a flow rate of 2 μl/min. The surface was then allowed to stabilise.

MCK data was obtained using bicyclic peptides of the invention as analytes injected at a flow rate of 30 μl/min to minimise any potential mass transfer effects. A five point, two-fold dilution range from 6.25 nM to 100 nM of analyte was prepared in running buffer. For each concentration, the association phases were monitored for 250 seconds and the dissociation phase was measured for 450 seconds. Regeneration of the sensor chip surface was conducted between cycles using the CAP Chip standard regeneration buffer (Cytiva). Multiple repeats of a blank and of Bicycle were programmed into the kinetic run in order to check the stability of both the surface and analyte over the kinetic cycles.

The signal from the reference channel F_{c}1 (no ACE2 captured) was subtracted from that of F_{c}2, F_{c}3 and F_{c}4 to correct for bulk effect and differences in non-specific binding to a reference surface. The signal from each blank run (F_{c}2-ACE2 captured but no antigen) was subtracted to correct for differences in surface stability. The double referenced sensorgrams were fitted with the Langmuir (1:1) binding model (Equation 1a above) where the closeness of fit of the data to the model is evaluated using the Chi square value which describes the deviation between the experimental and fitted (observed and expected) curves (Equation 1 b above).

TABLE 3
SPR MCK Direct Binding Assay Results for Selected
Bicyclic Peptides of the Invention

| BCY Number | Geometric Mean K_{D} (nM) |
|---|---|
| BCY15429 | 4.0 |
| BCY17158 | 8.1 |
| BCY17160 | 4.8 |
| BCY17161 | 4.4 |
| BCY17162 | 11.4 |
| BCY18210 | 10.6 |
| BCY18259 | 13.0 |
| BCY18549 | 42.9 |
| BCY18550 | 15.6 |
| BCY18551 | 19.5 |
| BCY18658 | 13.5 |
| BCY19024 | No sig. binding at top conc. tested |
| BCY19025 | 70.7 |
| BCY19026 | 45.2 |

1. A peptide ligand specific for ACE2 comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

2. The peptide ligand according to claim 1, wherein said loop sequences comprise 4, 5, 6 or 8 amino acids.

3. The peptide ligand according to claim 1 or claim 2, wherein said loop sequences comprise three reactive groups separated by two loop sequences one of which consists of 4 amino acids and the other of which consists of 8 amino acids, such as wherein the bicyclic peptide ligand comprises an amino acid sequence which is selected from:
(SEQ ID NO: 1)
CᵢHKFPCᵢᵢRDPQQYLFCᵢᵢᵢ;
(SEQ ID NO: 2)
CᵢTSPMCᵢᵢYVLKHQNRCᵢᵢᵢ;
(SEQ ID NO: 3)
CᵢTRPWCᵢᵢHSLLPRATCᵢᵢᵢ;
(SEQ ID NO: 4)
CᵢGRQFCᵢᵢHTLMPRHLCᵢᵢᵢ;
(SEQ ID NO: 5)
CᵢVRSHCᵢᵢSSLLPRIHCᵢᵢᵢ;
(SEQ ID NO: 9)
CᵢAPILCᵢᵢRWAERQGYCᵢᵢᵢ;
(SEQ ID NO: 10)
CᵢNAVLCᵢᵢSWARANSFCᵢᵢᵢ
(herein referred to as BCY17688);
(SEQ ID NO: 11)
CᵢNAVLCᵢᵢS[1Nal]ARANSFCᵢᵢᵢ;
(SEQ ID NO: 12)
CᵢNAVLCᵢᵢS[2Nal]ARANSFCᵢᵢᵢ;
(SEQ ID NO: 13)
CᵢNAVLCᵢᵢSW[Aib]RANSFCᵢᵢᵢ;
(SEQ ID NO: 14)
CᵢNAVLCᵢᵢSWA[HArg]ANSFCᵢᵢᵢ;
(SEQ ID NO: 19)
CᵢNAVLCᵢᵢSWA[Arg(Me)]ANSFCᵢᵢᵢ;
(SEQ ID NO: 20)
CᵢNKVLCᵢᵢSWASRNSMCᵢᵢᵢ;
(SEQ ID NO: 21)
CᵢNQVLCᵢᵢRWAQVNSMCᵢᵢᵢ;
(SEQ ID NO: 22)
CᵢNRVLCᵢᵢAWATANSMCᵢᵢᵢ;
(SEQ ID NO: 23)
CᵢNTVLCᵢᵢNWARYNSLCᵢᵢᵢ;
(SEQ ID NO: 24)
CᵢNTVLCᵢᵢAWARANSYCᵢᵢᵢ;
(SEQ ID NO: 25)
CᵢNTVLCᵢᵢGWARANSLCᵢᵢᵢ;
(SEQ ID NO: 26)
CᵢNTVLCᵢᵢAWAMNNSMCᵢᵢᵢ;
(SEQ ID NO: 27)
CᵢNTVLCᵢᵢAWATQNSLCᵢᵢᵢ;
(SEQ ID NO: 28)
CᵢSPVLCᵢᵢAWATRNSLCᵢᵢᵢ;
(SEQ ID NO: 29)
CᵢNA[tBuGly]LCᵢᵢSWARANSFCᵢᵢᵢ;
(SEQ ID NO: 30)
CᵢNA[tBuAla]LCᵢᵢSWARANSFCᵢᵢᵢ;
(SEQ ID NO: 31)
CᵢNAV[tBuGly]CᵢᵢSWARANSFCᵢᵢᵢ;
(SEQ ID NO: 32)
CᵢNAV[tBuAla]CᵢᵢSWARANSFCᵢᵢᵢ;
(SEQ ID NO: 33)
CᵢNAV[Cba]CᵢᵢSWARANSFCᵢᵢᵢ;
(SEQ ID NO: 34)
CᵢNAVLCᵢᵢSWARANS[2MePhe]Cᵢᵢᵢ;
(SEQ ID NO: 35)
CᵢNAVLCᵢᵢSWARANS[SMePhe]Cᵢᵢᵢ;
(SEQ ID NO: 36)
CᵢNAVLCᵢᵢSWARANS[MePhe]Cᵢᵢᵢ;
(SEQ ID NO: 37)
CᵢNAVLCᵢᵢSWARANS[2ClPhelCᵢᵢᵢ;
(SEQ ID NO: 38)
CᵢNAVLCᵢᵢSWARANS[3ClPhelCᵢᵢᵢ;
(SEQ ID NO: 39)
CᵢNAVLCᵢᵢSWARANS[4ClPhe]Cᵢᵢᵢ;
(SEQ ID NO: 40)
CᵢNAVLCᵢᵢSWARANS[2FPhe]Cᵢᵢᵢ;
(SEQ ID NO: 41)
CᵢNAVLCᵢᵢSWARANS[3FPhe]Cᵢᵢᵢ;
(SEQ ID NO: 42)
CᵢNAVLCᵢᵢSWA[Agb]ANSFCᵢᵢᵢ;
(SEQ ID NO: 43)
CᵢNAVLCᵢᵢSWARANSYCᵢᵢᵢ;
(SEQ ID NO: 44)
CᵢN[HyP]VLCᵢᵢSWA[HArg]ANSFCᵢᵢᵢ;
(SEQ ID NO: 45)
CᵢNAVLCᵢᵢSWA[HArg][dA]NSFCᵢᵢᵢ;
(SEQ ID NO: 46)
CᵢN[dD]VLCᵢᵢSWA[HArg]ANSFCᵢᵢᵢ;
(SEQ ID NO: 47)
CᵢN[dA]VLCᵢᵢSWA[HArg]ANSFCᵢᵢᵢ;
(SEQ ID NO: 48)
CᵢNRVLCᵢᵢAWATANS[Hse(Me)]Cᵢᵢᵢ;
(SEQ ID NO: 49)
CᵢN[HArg]VLCᵢᵢSWA[HArg]ANSFCᵢᵢᵢ;
(SEQ ID NO: 50)
CᵢNTVLCᵢᵢGWA[HArg]ANSLCᵢᵢᵢ;
(SEQ ID NO: 51)
CᵢNTVLCᵢᵢGWARANSLCᵢᵢᵢ;
(SEQ ID NO: 52)
CᵢNTVLCᵢᵢSWA[HArg]ANSFCᵢᵢᵢ;
(SEQ ID NO: 53)
CᵢNSVLCᵢᵢSWA[HArg]ANSFCᵢᵢᵢ;
(SEQ ID NO: 54)
CᵢNDVLCᵢᵢSWA[HArg]ANSFCᵢᵢᵢ;
(SEQ ID NO: 55)
CᵢNEVLCᵢᵢSWA[HArg]ANSFCᵢᵢᵢ;
(SEQ ID NO: 56)
CᵢNAVLCᵢᵢSWA[HArg][HyP]NSFCᵢᵢᵢ;
(SEQ ID NO: 57)
CᵢNAVLCᵢᵢSWA[HArg]TNSFCᵢᵢᵢ;
and
(SEQ ID NO: 58)
CᵢNAVLCᵢᵢSWA[HArg]SNSFCᵢᵢᵢ;
wherein Cᵢ, Cᵢᵢ and Cᵢᵢᵢ represent first, second and third cysteine residues, respectively, 1Nal represents 1-naphthylalanine, 2Nal represents 2-naphthylalanine, Aib represents aminoisobutyric acid, HArg represents homoarginine, Arg(Me) represents δ-N methyl arginine, tBuGly represents t-butyl-glycine, tBuAla represents t-butyl-alanine, Cba represents β-cyclobutylalanine, 2MePhe represents 2-methyl-phenylalanine, 3MePhe represents 3-methyl-phenylalanine, 4MePhe represents 4-methyl-phenylalanine, 2ClPhe represents 2-chloro-phenylalanine, 3ClPhe represents 3-chloro-phenylalanine, 4ClPhe represents 4-chloro-phenylalanine, 2FPhe represents 2-fluoro-phenylalanine, 3FPhe represents 3-fluoro-phenylalanine, Agb represents 2-amino-4-guanidinobutyric acid, HyP represents hydroxyproline and Hse(Me) represents homoserine-methyl, or a pharmaceutically acceptable salt thereof;
or wherein the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15296);
A-(SEQ ID NO: 2)-A (herein referred to as BCY15295);
A-(SEQ ID NO: 3)-A (herein referred to as BCY15293);
A-(SEQ ID NO: 4)-A (herein referred to as BCY15292);
A-(SEQ ID NO: 5)-A (herein referred to as BCY15291);
A-(SEQ ID NO: 9)-A (herein referred to as BCY15425);
A-(SEQ ID NO: 10)-A (herein referred to as BCY15429); A-(SEQ ID NO: 10)-A-[K(PYA)] (herein referred to as BCY17344);
Ac-(SEQ ID NO: 10) (herein referred to as BCY17663);
Ac-A-(SEQ ID NO: 10)-A (herein referred to as BCY17691);
Ac-(SEQ ID NO: 14) (herein referred to as BCY18259);
Ac-(SEQ ID NO: 14)-[K(PYA)] (herein referred to as BCY18784);
A-(SEQ ID NO: 20)-A (herein referred to as BCY17157);
A-(SEQ ID NO: 21)-A (herein referred to as BCY17158);
A-(SEQ ID NO: 22)-A (herein referred to as BCY17159);
A-(SEQ ID NO: 23)-A (herein referred to as BCY17160);
A-(SEQ ID NO: 24)-A (herein referred to as BCY17161);
A-(SEQ ID NO: 25)-A (herein referred to as BCY17162);
A-(SEQ ID NO: 26)-A (herein referred to as BCY17163);
A-(SEQ ID NO: 27)-A (herein referred to as BCY17164);
A-(SEQ ID NO: 28)-A (herein referred to as BCY17165);
A-(SEQ ID NO: 29)-A (herein referred to as BCY17330);
A-(SEQ ID NO: 30)-A (herein referred to as BCY17331);
A-(SEQ ID NO: 31)-A (herein referred to as BCY17332);
A-(SEQ ID NO: 32)-A (herein referred to as BCY17333);
A-(SEQ ID NO: 33)-A (herein referred to as BCY17334);
A-(SEQ ID NO: 34)-A (herein referred to as BCY17335);
A-(SEQ ID NO: 35)-A (herein referred to as BCY17336);
A-(SEQ ID NO: 36)-A (herein referred to as BCY17337);
A-(SEQ ID NO: 37)-A (herein referred to as BCY17338);
A-(SEQ ID NO: 38)-A (herein referred to as BCY17339);
A-(SEQ ID NO: 39)-A (herein referred to as BCY17340);
A-(SEQ ID NO: 40)-A (herein referred to as BCY17341);
A-(SEQ ID NO: 41)-A (herein referred to as BCY17342);
A-(SEQ ID NO: 42)-A (herein referred to as BCY17689);
A-(SEQ ID NO: 43)-A (herein referred to as BCY17690);
Ac-(SEQ ID NO: 44) (herein referred to as BCY18210);
Ac-(SEQ ID NO: 45) (herein referred to as BCY18256);
Ac-(SEQ ID NO: 46) (herein referred to as BCY18257);
Ac-(SEQ ID NO: 47) (herein referred to as BCY18258);
Ac-(SEQ ID NO: 48)-[K(PYA)] (herein referred to as BCY18349);
Ac-(SEQ ID NO: 49) (herein referred to as BCY18350);
Ac-(SEQ ID NO: 50)-A-[K(PYA)] (herein referred to as BCY18549);
Ac-(SEQ ID NO: 50)-[K(PYA)] (herein referred to as BCY18551);
Ac-(SEQ ID NO: 51)-[K(PYA)] (herein referred to as BCY18550);
Ac-(SEQ ID NO: 52) (herein referred to as BCY18657);
Ac-(SEQ ID NO: 53) (herein referred to as BCY18658);
Ac-(SEQ ID NO: 54) (herein referred to as BCY18659);
Ac-(SEQ ID NO: 55) (herein referred to as BCY18783);
Ac-(SEQ ID NO: 56) (herein referred to as BCY19024);
Ac-(SEQ ID NO: 57) (herein referred to as BCY19025); and
Ac-(SEQ ID NO: 58) (herein referred to as BCY19026);
wherein PYA represents propargyl-acid.
or wherein the molecular scaffold is TATA, the bicyclic peptide additionally comprises N- and/or C-terminal additions and a labelling moiety, such as fluorescein (FI), and comprises an amino acid sequence which is selected from:
A-(SEQ ID NO: 1)-A-[Sar₆]-[KFI] (herein referred to as BCY15288);
A-(SEQ ID NO: 2)-A-[Sar₆]-[KFI] (herein referred to as BCY15287);
A-(SEQ ID NO: 3)-A-[Sar₆]-[KFI] (herein referred to as BCY15285);
A-(SEQ ID NO: 4)-A-[Sar₆]-[KFI] (herein referred to as BCY15284);
A-(SEQ ID NO: 5)-A-[Sar₆]-[KFI] (herein referred to as BCY15283);
A-(SEQ ID NO: 9)-A-[Sar₆]-[KFI] (herein referred to as BCY15419);
A-(SEQ ID NO: 10)-A-[Sar₆]-[KFI] (herein referred to as BCY15423);
A-(SEQ ID NO: 11)-A-[Sar₆]-[KFI] (herein referred to as BCY16866);
A-(SEQ ID NO: 12)-A-[Sar₆]-[KFI] (herein referred to as BCY16867);
A-(SEQ ID NO: 13)-A-[Sar₆]-[KFI] (herein referred to as BCY16872);
A-(SEQ ID NO: 14)-A-[Sar₆]-[KFI] (herein referred to as BCY16874); and
A-(SEQ ID NO: 19)-A-[Sar₆]-[KFI] (herein referred to as BCY16863).

4. The peptide ligand according to claim 1 or claim 2, wherein said loop sequences comprise three reactive groups separated by two loop sequences one of which consists of 5 amino acids and the other of which consists of 4 amino acids, such as wherein the bicyclic peptide ligand comprises an amino acid sequence which is selected from:
(SEQ ID NO: 15)
CᵢLELYQCᵢWRGKCᵢᵢ;
(SEQ ID NO: 16)
CᵢPSQYKCᵢᵢWRGKCᵢᵢᵢ;
(SEQ ID NO: 17)
CᵢLEVYKCᵢᵢWRGKCᵢᵢᵢ;
(SEQ ID NO: 59)
CᵢAEIYKCᵢᵢWRGRCᵢᵢᵢ;
(SEQ ID NO: 60)
CᵢDTLYKCᵢᵢWRGRCᵢᵢᵢ;
(SEQ ID NO: 61)
CᵢESLYKCᵢᵢWRGRCᵢᵢᵢ;
(SEQ ID NO: 62)
CᵢNTLYKCᵢᵢWRGKCᵢᵢᵢ;
and
(SEQ ID NO: 63)
CᵢTELYKCᵢᵢWRGRCᵢᵢᵢ;
wherein Cᵢ, Cᵢᵢ and Cᵢᵢᵢ represent first, second and third cysteine residues, respectively, or a pharmaceutically acceptable salt thereof;
or wherein the molecular scaffold is TATA and the bicyclic peptide ligand additionally comprises N- and/or C-terminal additions and comprises an amino acid sequence which is selected from:
A-(SEQ ID NO: 15)-A (herein referred to as BCY15426);
A-(SEQ ID NO: 16)-A (herein referred to as BCY15427);
A-(SEQ ID NO: 17)-A (herein referred to as BCY15428);
A-(SEQ ID NO: 59)-A (herein referred to as BCY17152);
A-(SEQ ID NO: 60)-A (herein referred to as BCY17153);
A-(SEQ ID NO: 61)-A (herein referred to as BCY17154);
A-(SEQ ID NO: 62)-A (herein referred to as BCY17155); and
A-(SEQ ID NO: 63)-A (herein referred to as BCY17156);
or wherein the molecular scaffold is TATA, the bicyclic peptide additionally comprises N- and/or C-terminal additions and a labelling moiety, such as fluorescein (FI), and comprises an amino acid sequence which is:
A-(SEQ ID NO: 15)-A-[Sar₆]-[KFI] (herein referred to as BCY15420);
A-(SEQ ID NO: 16)-A-[Sar₆]-[KFI] (herein referred to as BCY15421); and
A-(SEQ ID NO: 17)-A-[Sar₆]-[KFI] (herein referred to as BCY15422).

5. The peptide ligand according to claim 1 or claim 2, wherein said loop sequences comprise three reactive groups separated by two loop sequences one of which consists of 5 amino acids and the other of which consists of 8 amino acids, such as wherein the bicyclic peptide ligand comprises an amino acid sequence which is selected from:
(SEQ ID NO: 18)
CᵢAN[Aᵢb]VLCᵢᵢSWARANSFCᵢᵢᵢ;
wherein Cᵢ, Cᵢᵢ and Cᵢᵢᵢ represent first, second and third cysteine residues, respectively, Aib represents aminoisobutyric acid, or a pharmaceutically acceptable salt thereof;
or wherein the molecular scaffold is TATA and the bicyclic peptide ligand additionally comprises N- and/or C-terminal additions, and a labelling moiety, such as fluorescein (FI), and comprises an amino acid sequence which is:
A-(SEQ ID NO: 18)-A-[Sar₆]-[KFI] (herein referred to as BCY16871).

6. The peptide ligand according to claim 1 or claim 2, wherein said loop sequences comprise three reactive groups separated by two loop sequences one of which consists of 6 amino acids and the other of which consists of 4 amino acids, such as wherein the bicyclic peptide ligand comprises an amino acid sequence which is selected from:
(SEQ ID NO: 6)
CᵢGREELPCᵢᵢRIKLCᵢᵢᵢ;
and
(SEQ ID NO: 7)
CᵢLRSYNLCᵢᵢPRINCᵢᵢᵢ;
wherein Cᵢ, Cᵢᵢ and Cᵢᵢᵢ represent first, second and third cysteine residues, respectively, or a pharmaceutically acceptable salt thereof;
or wherein the molecular scaffold is TATA, the bicyclic peptide ligand additionally comprises N- and/or C-terminal additions and comprises an amino acid sequence which is selected from:
A-(SEQ ID NO: 6)-A (herein referred to as BCY15298); and
A-(SEQ ID NO: 7)-A (herein referred to as BCY15294);
or wherein the molecular scaffold is TATA, the bicyclic peptide additionally comprises N- and/or C-terminal additions and a labelling moiety, such as fluorescein (FI), and comprises an amino acid sequence which is selected from:
A-(SEQ ID NO: 6)-A-[Sar₆]-[KFI] (herein referred to as BCY15290); and
A-(SEQ ID NO: 7)-A-[Sar₆]-[KFI] (herein referred to as BCY15286).

7. The peptide ligand according to claim 1 or claim 2, wherein said loop sequences comprise three reactive groups separated by two loop sequences both of which consist of 6 amino acids, such as wherein the bicyclic peptide ligand comprises an amino acid sequence which is:
(SEQ ID NO: 8)
CᵢHRDFPRCᵢᵢTWETQWCᵢᵢᵢ;
wherein Cᵢ, Cᵢᵢ and Cᵢᵢᵢ represent first, second and third cysteine residues, respectively, or a pharmaceutically acceptable salt thereof;
or wherein the molecular scaffold is TATA and the bicyclic peptide ligand additionally comprises N- and/or C-terminal additions and comprises an amino acid sequence which is:
A-(SEQ ID NO: 8)-A (herein referred to as BCY15297);
or wherein the molecular scaffold is TATA, the bicyclic peptide additionally comprises N- and/or C-terminal additions and a labelling moiety, such as fluorescein (FI), and comprises an amino acid sequence which is:
A-(SEQ ID NO: 8)-A-[Sar₆]-[KFI] (herein referred to as BCY15289).

8. The peptide ligand according to any one of claims 1 to 7, wherein the pharmaceutically acceptable salt is selected from the free acid or the sodium, potassium, calcium and ammonium salt.

9. A pharmaceutical composition which comprises the peptide ligand of any one of claims 1 to 8, in combination with one or more pharmaceutically acceptable excipients.

10. The pharmaceutical composition according to claim 9, which additionally comprises one or more therapeutic agents.

11. The peptide ligand according to any of claims 1 to 8, or the pharmaceutical composition as defined in claim 9 or claim 10, for use in the prophylaxis or treatment of a respiratory disorder.

12. The peptide ligand for use according to claim 11, wherein the inflammatory response is mediated by a viral infection, such as an infection of: rhinovirus; respiratory syncytial virus (RSV); human metapneumovirus (hMPV); influenza; severe acute respiratory syndrome coronavirus (SARS-CoV or SARS-CoV-1); severe acute respiratory syndrome-related coronavirus (SARSr-CoV); severe acute respiratory syndrome coronavirus 2 (SARS-CoV-2); or Middle East respiratory syndrome coronavirus (MERS-CoV), in particular infection of severe acute respiratory syndrome coronavirus 2 (SARS-CoV-2).

13. The peptide ligand for use according to claim 11, wherein the respiratory disorder is selected from: Coronavirus disease 2019 (COVID-19), severe acute respiratory syndrome (SARS), Middle East respiratory syndrome (MERS), acute lung injury (ALI), acute respiratory distress syndrome (ARDS) and pulmonary arterial hypertension (PAH), such as Coronavirus disease 2019 (COVID-19).
